SEQUENCE LISTING (6 sequences)

SEQ ID NO: 1 - protein, 1042 aa, Homo sapiens
MPGIVVFRRRWSVGSDDLVLPAIFLFLLHTTWFVILSVVLFGLVYNPHEACSLNLVDHGRGYLGILLSCMIAEMAIIWLSMRGGILYTEPRDSMQYVLYVRLAILVIEFIYAIVGIVWLTQYYTSCNDLTAKNVTLGMVVCNWVVILSVCITVLCVFDPTGRTFVKLRATKRRQRNLRTYNLRHRLEEGQATSWSRRLKVFLCCTRTKDSQSDAYSEIAYLFAEFFRDLDIVPSDIIAGLVLLRQRQRAKRNAVLDEANNDILAFLSGMPVTRNTKYLDLKNSQEMLRYKEVCYYMLFALAAYGWPMYLMRKPACGLCQLARSCSCCLCPARPRFAPGVTIEEDNCCGCNAIAIRRHFLDENMTAVDIVYTSCHDAVYETPFYVAVDHDKKKVVISIRGTLSPKDALTDLTGDAERLPVEGHHGTWLGHKGMVLSAEYIKKKLEQEMVLSQAFGRDLGRGTKHYGLIVVGHSLGAGTAAILSFLLRPQYPTLKCFAYSPPGGLLSEDAMEYSKEFVTAVVLGKDLVPRIGLSQLEGFRRQLLDVLQRSTKPKWRIIVGATKCIPKSELPEEVEVTTLASTRLWTHPSDLTIALSASTPLYPPGRIIHVVHNHPAEQCCCCEQEEPTYFAIWGDNKAFNEVIISPAMLHEHLPYVVMEGLNKVLENYNKGKTALLSAAKVMVSPTEVDLTPELIFQQQPLPTGPPMPTGLALELPTADHRNSSVRSKSQSEMSLEGFSEGRLLSPVVAAAARQDPVELLLLSTQERLAAELQARRAPLATMESLSDTESLYSFDSRRSSGFRSIRGSPSLHAVLERDEGHLFYIDPAIPEENPSLSSRTELLAADSLSKHSQDTQPLEAALGSGGVTPERPPSAAANDEEEEVGGGGGGPASRGELALHNGRLGDSPSPQVLEFAEFIDSLFNLDSKSSSFQDLYCMVVPESPTSDYAEGPKSPSQQEILLRAQFEPNLVPKPPRLFAGSADPSSGISLSPSFPLSSSGELMDLTPTGLSSQECLAADKIRTSTPTGHGASPAKQDELVISAR

SEQ ID NO: 2 - DNA, 5687 bp, Homo sapiens
ggccttggggagcccaggatggaggtggcggtcgcggcggcgggccgagccctgcggcgggcgggaggaggtgagcaccaggcccactgagcctctgcagagccaccagccatgcccgggatcgtggtgttccggcggcgctggtctgtgggcagtgatgacctcgtcctaccagccatcttcctctttctcctgcataccacctggtttgtgatcctgtccgtggtgctcttcggcctggtctataacccgcacgaggcctgctccctgaacctggtggaccacggccgcggctacctgggcatcctgctgagctgcatgatcgctgagatggccatcatctggctgagcatgcgcgggggcatcctctacacggagccccgtgactccatgcagtacgtgctctacgtgcgcctggccatcctggtgatcgagttcatctacgccatcgtgggcatcgtctggctcactcagtactacacctcctgcaacgacctcactgccaagaatgtcaccctcggaatggttgtctgcaactgggtagtcatcctcagtgtgtgcatcactgtcctctgcgtcttcgaccccacgggccgcacctttgtcaagctgagagccaccaagaggaggcagcgtaacctgcggacctacaacctgcggcaccgcttagaggagggtcaagccaccagctggtcgcgccggctcaaagtgttcctctgctgcacgcggacgaaggactcccagtcagatgcctactcagaaatcgcctacctctttgcggagttcttccgggaccttgacattgtgccatccgacatcattgctggcctggtgctgctccggcagcggcagcgggccaagcgcaacgccgtgctggacgaggcaaacaatgacatcttggccttcctgtctgggatgccggtgaccagaaacaccaagtacctcgacctcaagaattcacaagagatgctccgctacaaagaggtctgctactacatgctctttgccctggctgcctacgggtggcccatgtacctgatgcggaagcccgcctgcggcctctgccaactggctcggtcctgctcgtgttgcctgtgtcctgcgaggccgcggttcgcccctggagtcaccatcgaggaagacaactgctgtggctgtaatgccattgccatccggcgccacttcctggacgagaacatgactgcggtggacatcgtctatacctcctgccatgatgcggtctatgaaacgcccttctacgtggcggtggaccatgacaagaagaaagtggtgatcagtatccgggggaccctgtcccccaaggatgccctgactgacctgacgggtgatgctgagcgcctccccgtggaggggcaccacggcacctggctgggccacaagggtatggtcctctcagctgagtacatcaagaagaaactggagcaggagatggtcctgtcccaggcctttgggcgagacctgggccgcggaaccaaacactacggcctgattgtggtgggccactccctgggcgcgggcactgctgccatcctctccttccttctgcgcccacagtatccgaccctcaagtgctttgcctactccccgccagggggcctgctgagtgaggatgcaatggagtattccaaggagttcgtgactgctgtggttctgggcaaagacctcgtccccaggattggcctctctcagctggaaggcttccgcagacagctcctggatgtcctgcagcgaagcaccaagcccaaatggcggatcatcgtgggggccaccaaatgcatccccaagtcggagctgcctgaggaggtagaggtgaccaccctggccagcacgcggctctggacccaccccagcgacctaactatagccctctcagccagcactccactctacccgcccggccgcatcatccacgtggtccacaaccaccctgcagagcagtgctgctgctgtgagcaggaggagcccacatactttgccatctggggcgacaacaaggccttcaatgaggtgatcatctcgccagccatgctgcatgagcacctgccctatgtggtcatggaggggctcaacaaggtgctggagaactacaacaaggggaagaccgctctgctctctgcagccaaggtcatggtgagccctaccgaggtggacctgactcctgagctcatcttccagcagcagccactccccacggggccgcccatgcccactggccttgccctggagctgccgactgcagaccaccgcaacagcagcgtcaggagcaagtcccagtctgagatgagcctggagggcttctcggaggggcggctgctgtcgccagtggttgcggcggcggcccgccaggacccggtggagctgctgctgctgtctacccaggagcggctggcggcggagctgcaggcccggcgggcaccactggccaccatggagagcctctcggacactgagtccctgtacagcttcgactcgcgccgctcctcaggcttccgcagcatccggggctcccccagcctccacgctgtgctggagcgtgatgaaggccacctcttctacattgaccctgccatccccgaggaaaacccatccctgagctcgcgcactgagctgctggcggccgacagcctgtccaagcactcacaggacacgcagcccctggaggcggccctgggcagtggcggcgtcactcctgagcggccccccagtgctgcggccaatgacgaggaggaagaggttggcggtgggggtggcgggccggcctcccgcggggagctggcgctgcacaatgggcgcctgggggactcgcccagtcctcaggtgctggaattcgccgagttcatcgacagcctcttcaacctggacagcaagagcagctccttccaagacctctactgcatggtggtgcccgagagccccaccagtgactacgctgagggccccaagtcccccagccagcaagagatcctgctccgtgcccagttcgagcccaacctggtgcccaagcccccacggctctttgccggctcagccgacccctcctcgggcatctcactctcgccctccttcccgctcagctcctcgggtgagctcatggacctgacgcccacgggcctcagtagccaggaatgcctggcggctgacaagatccggacttctacccccactggccacggagccagccccgccaagcaagatgagctggtcatctcagcacgctagcaccccagttgcgtggccagccgggcccaggcaggagcaggtggccctgtgggcacctggtgcctgccccctgccgggcagctttaaggacagacccccaggggcagtttagcctcaggcacaggcatcgctgctgagctgggggtccgcatccctacctcagcttaggacccccagagccaaggtggctgggatctggccccacagatggggaaagatggggaagggtgtggagtggggaggagcctgggcagcctgctgggtgggccacactcagcctgactgccctccatgggggcattctggcaccccctgctccaggacaggccatgggcaagctgcctcccatcactgcctgctggctgctctcccaggggccaggtgtgaagagcagtgccccccgacacatgtattctcatctgtggtccaggccggcatcgtcctggccaccccccagatctggtgcctgctggccggccccctggggtgcccctgccgaggtggcctgcagtgctgtacatgtttacagaagctgctgggcttggctcaggatgtgttctgggcttgcaagccccccgcccaatcatgtgttcagtagccgtcctctgagcagggcccaaggcagccaggggcctggaggggccagaggagggtggggtcagggccgccccttctctgccttgtgcctctcatgctgcctcctctgcccatgggtcctgggcacccaggcctgccctgcctgctggctacttcctggcttaccttctacccccaaggatcctcaccacccaaagggtggtgggcactgctgtgaccaccccagctgcagagtcagtgccctgggtggaaggaaggcactgagagcccccttcctctgagggccccacctcaccccttggtgtcacccccaccacgcctaggcagctctgggccctgggatctggaaccaacacacccctgttcccctcagctttccctcctcgctggcctgggcaccctcctgggagcaggccttcctccctcccacccccaatgtcctgttggtaggaggtggggccaagagtggggtatggtgggccttggctggagacctctgtccactgcccagggaggggcctggggctgggagcagtcccggtttagcctgaggtccccatagggcttcctcccctgctgggtttgggaagcagttagggagatagcgacccggagtttccccagaagcggggtgggagggtgtgcatgctagtgttggcgcgtatgcatgtgcatgagtgtgcaccgttcctaaggaaggggcctctggggctgcccaccctacctgccctgcctgcctgctgcccctcccagcctgccaagaaaacggtaggggagcatgatggggcctttgaggcagggtcgcagggacaagctcagctttaggcaccatctgttcccatcacgcctgctgctgtgacccgttttggaaaactggtgtgtaccgaggcgctgactgcacggctgaccgcctgctcgtgccttcattctgcagcggcatggtccctcccattctggctccacctgcagcctccctgggtggcctaggctcccccgaccaagagacctccctctcatgatcactggtacctgggggcctgaattctggcccccggctccccacacagctgggactggcctggatggctgtcctgggagcccctgcccaccctgacggagctgggcctcccctcatcctctgtaactcccgccttcaccagactcaaggacaccctggccctgctgaggcatacagagcttcagcccagcacagaagcaagacaaaatcagtggctcttagagtttagaaaacaagacagactctcagatgaaagatctgacaagcaccgtggccagtcacagggagagacttgatgtctggccttttaattcctcctctgccagggtgggtcctgggacctctaatgtgggcatgtcgtccaccccaggacgagccatcagggacagaccccccacccccaaggctgcagccacaccatgtttcaggcttggggctggggcaggcttgggctcaatcctgggcacccaggggcagcccacccctaacctggctcctacccaccttgcccttgaaggatgggcctgctgcacgtctccctcctccaccccataccacactggggggtctgagccacccccctcagccccgttcggctcagaccgacccccactccatccccagacctgcagcacaagtgcgcgggcctgtcctcccaggggcctgggcgactccatatgcaatcagtagcgagcagccgggccccacagaccctcatgcactctcttacgtgccattctccccagactttttttgtacttaatgtatgaaagatccaaactaatattgctgtaaaaaggagagacaaattaatatagcttattctataaat

SEQ ID NO: 3 - DNA, 22 bp, Homo sapiens
cgacctcactgccaagaatgtc

SEQ ID NO: 4 - DNA, 22 bp, Homo sapiens
cttgaccctcctctaagcggtg

SEQ ID NO: 5 - DNA, 38 bp, Homo sapiens
ccataagcttgccaccatgcccgggatcgtggtgttcc

SEQ ID NO: 6 - DNA, 34 bp, Homo sapiens
ccatggtaccgagcgtgctgagatgaccagctca

(19) United States
(12) Patent Application Publication
(10) Pub. No.: US 2007/0142276 A1
(43) Pub. Date: Jun. 21, 2007

(54) Protein involved in carcinoma

(76) Inventor: Alasdair Craig Stamps, Berkshire (GB)

Correspondence Address: KLAUBER & JACKSON, 411 HACKENSACK AVENUE, HACKENSACK, NJ, 07601, US

(21) Appl. No.: 10/569,224

(22) PCT Filed: Aug. 27, 2004
(86) PCT No.: PCT/GB04/03643

§ 371 Date: Jan. 29, 2007

(30) Foreign Application Priority Data
Sep. 5, 2003 (GB) 0320878.2

(51) Int. Cl. A61K 38/17

(52) U.S. Cl. 514/12

(57) ABSTRACT

The present invention provides a polypeptide, KIAA0659, of use in the treatment and/or prophylaxis of carcinoma. Also provided are agents which interact with or modulate the expression or activity of the polypeptide, methods for the identification of such agents and the use of KIAA0659 in the diagnosis of carcinoma.

The present invention relates to methods for the treatment and/or prophylaxis of carcinoma comprising targeting of a KIAA0659 polypeptide, agents which interact with or modulate the expression or activity of the polypeptide, methods for the identification of such agents and the use of KIAA0659 in the diagnosis of carcinoma, for example, lung and/or kidney and/or liver cancer.

Cancer of the kidney, sometimes referred to as renal cell carcinoma, is the most common tumour arising from the kidney. At present, surgery is the main treatment for kidney cancer with radiotherapy being sometimes used instead of surgery for patients who are too ill to undergo a major operation, however, renal cell kidney cancers are not particularly sensitive to radiotherapy. The two most common biological treatments for kidney cancer are the use of the cytokines, interleukin-2 and interferon-alpha, which are used in an attempt to stimulate the immune system of the patient.

Lung cancer is a leading cancer killer in both men and women with two major types: non-small cell lung cancer and small cell lung cancer. Treatment is restricted to surgery where possible, and chemotherapy and radiotherapy. Thus, the challenge is to develop better means to detect cancer early, to identify persons with premalignant disease so they can be monitored more closely and treated with chemopreventive drugs and to develop better therapies to treat lung cancer.

There are two main types of liver cancer; hepatoma, also known as hepatocellular carcinoma, is the most common type of primary liver cancer and accounts for around 85% of all primary liver cancers. It develops from the main liver cells called hepatocytes. Cholangiocarcinoma arises in the cells that line the bile duct and it accounts for around 12% of primary liver cancers. The main treatments for primary liver cancer are surgery and chemotherapy with surgical removal considered to be the most effective treatment. Unfortunately, about 70% of patients cannot have this surgery due the size or location of the tumours or other health factors. Thus, important needs exist for new therapeutic agents for the treatment of liver cancer. Additionally, there is a clear need to identify new carcinoma-associated proteins for use as sensitive and specific biomarkers for the diagnosis of carcinoma in living subjects.

No previous literature discloses the involvement of KIAA0659 polypeptide expression or activity in carcinoma. The present invention is based on the finding that KIAA0659 represents a novel therapeutic target for the treatment and/or prophylaxis of carcinoma.

Accordingly, the invention provides a method for the treatment and/or prophylaxis of carcinoma comprising administering a therapeutically effective amount of an agent which interacts with or modulates the expression or activity of a KIAA0659 polypeptide.

A KIAA0659 polypeptide includes a polypeptide which:

(a) comprises or consists of the amino acid sequence of SEQ ID NO:1; or

(b) is a derivative having one or more amino acid substitutions, modifications, deletions or insertions relative to the amino acid sequence of SEQ ID NO:1 which retains the activity of KIAA0659.

The term “polypeptides” includes peptides, polypeptides and proteins. These are used interchangeably unless otherwise specified.

In the present application, the term “carcinoma” includes a malignant new growth that arises from epithelium, found in skin or, more commonly, the lining of body organs, for example: breast, prostate, lung, kidney, pancreas, stomach, bowel or bladder. Carcinomas tend to infiltrate into adjacent tissue and spread (metastasise) to distant organs, for example: to bone, liver, lung or the brain. In one embodiment, the carcinoma is kidney cancer. In another embodiment, the carcinoma is liver cancer and in yet another embodiment, the carcinoma is lung cancer.

Agents of use in the methods of the invention include without limitation, agents that are capable of interacting with (e.g. binding to, or recognising) a KIAA0659 polypeptide or a nucleic acid molecule encoding a KIAA0659 polypeptide, or are capable of modulating the interaction, expression, activity of a KIAA0659 polypeptide or the expression of a nucleic acid molecule encoding a KIAA0659 polypeptide. Such agents include, without limitation, antibodies, nucleic acids (e.g. DNA and RNA), carbohydrates, lipids, proteins, polypeptides, peptides, peptidomimetics, small molecules and other drugs.

Thus, the invention also provides the use of an agent, which interacts with or modulates the expression or activity of a KIAA0659 polypeptide for the manufacture of a medicament for the treatment and/or prophylaxis of carcinoma.

Thus, the invention also provides the use of an agent, which interacts with or modulates the expression or activity of a KIAA0659 polypeptide in the manufacture of a medicament for the treatment and/or prophylaxis of carcinoma.

Most preferably, the agent for use in the treatment and/or prophylaxis of carcinoma is an antibody that interacts with (i.e. binds to or recognises) or modulates the activity of a KIAA0659 polypeptide. Accordingly, there is provided the use of an antibody that interacts with a KIAA0659 polypeptide for use in the manufacture of a medicament for use in the treatment and/or prophylaxis of carcinoma. Also provided is a method of treatment and/or prophylaxis of carcinoma in a subject comprising administering to said subject a therapeutically effective amount of an antibody that interacts with KIAA0659. In one embodiment, an antibody that interacts with a KIAA0659 polypeptide may be used to mediate antibody dependent cell cytotoxicity (ADCC) and/or complement dependent cytotoxicity (CDC). In such a case the antibody is preferably a full-length naked antibody. In another aspect of the invention, an antibody that interacts with KIAA0659 polypeptides may be used to inhibit the activity of said polypeptides.

Most preferred are antibodies that specifically interact with a KIAA0659 polypeptide. Specifically interacting with (e.g. recognising or binding to) means that the antibodies have a greater affinity for KIAA0659 polypeptides than for other polypeptides.

An antibody, optionally conjugated to a therapeutic moiety, can be used therapeutically alone or in combination with a cytotoxic factor(s) and/or cytokine(s). In particular, KIAA0659 antibodies can be conjugated to a therapeutic agent, such as a cytotoxic agent, a radionuclide or drug moiety to modify a given biological response. The therapeutic agent is not to be construed as limited to classical chemical therapeutic agents. For example, the therapeutic agent may be a drug moiety which may be a protein or polypeptide possessing a desired biological activity. Such moieties may include, for example and without limitation, a toxin such as abrin, ricin A, pseudomonas exotoxin, or diphtheria toxin, a protein such as tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Therapeutic agents also include cytotoxins or cytotoxic agents including any agent that is detrimental to (e.g. kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other therapeutic moieties may include radionuclides such as Indium¹¹¹ and Yttrium⁹⁰, Lutetium¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin.

Techniques for conjugating such therapeutic agents to antibodies are well known in the art (see, e.g. Arnon et al., “Monoclonal Antibodies For Immunotargeting Of Drugs In Cancer Therapy”, in Monoclonal Antibodies And Cancer Therapy, Reisfeld et al., eds., 1985 pp. 243-56, ed. Alan R. Liss, Inc; Hellstrom et al., “Antibodies For Drug Delivery”, in Controlled Drug Delivery, 2nd Ed., Robinson et al., eds., 1987, pp. 623-53, Marcel Dekker, Inc.; Thorpe, “Antibody Carriers Of Cytotoxic Agents In Cancer Therapy: A Review”, in Monoclonal Antibodies '84: Biological And Clinical Applications; Pinchera et al., 1985, eds., pp. 475-506; “Analysis, Results, And Future Prospective Of The Therapeutic Use Of Radiolabelled Antibody In Cancer Therapy”, in Monoclonal Antibodies For Cancer Detection And Therapy, Baldwin et al. (eds.), 1985, pp. 303-16, Academic Press; Thorpe et al., 1982 “The Preparation And Cytotoxic Properties Of Antibody-Toxin Conjugates”, Immunol. Rev., 62:119-58 and Dubowchik et al., 1999, Pharmacology and Therapeutics, 83, 67-123).

The antibodies for use in the invention include analogues and derivatives that are modified, for example but without limitation, by the covalent attachment of any type of molecule. Preferably, said attachment does not impair immunospecific binding. In one aspect, an antibody can be conjugated to a second antibody to form an antibody heteroconjugate (see U.S. Pat. No. 4,676,980).

In other embodiments, the invention provides the therapeutic use of fusion proteins of the antibodies (or functionally active fragments thereof), for example but without limitation, where the antibody or fragment thereof is fused via a covalent bond (e.g. a peptide bond), at optionally the N-terminus or the C-terminus, to an amino acid sequence of another protein (or portion thereof; preferably at least a 10, 20 or 50 amino acid portion of the protein). Preferably the antibody, or fragment thereof, is linked to the other protein at the N-terminus of the constant domain of the antibody. In another aspect, an antibody fusion protein may facilitate depletion or purification of a polypeptide as described herein, increase half-life in vivo, and enhance the delivery of an antigen across an epithelial barrier to the immune system.

Where the fusion protein is an antibody fragment linked to an effector or reporter molecule, this may be prepared by standard chemical or recombinant DNA procedures. A preferred effector group is a polymer molecule, which may be attached to the modified Fab fragment to increase its half-life in vivo.

The polymer molecule may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Particular optional substituents which may be present on the above-mentioned synthetic polymers include one or more hydroxy, methyl or methoxy groups. Particular examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Particular naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, preferably from 5000 to 40000 Da and more preferably from 25000 to 40000 Da. The polymer size may in particular be selected on the basis of the intended use of the product. Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 25000 Da to 40000 Da.

Particularly preferred polymers include a polyalkylene polymer, such as a poly(ethyleneglycol) or, especially, a methoxypoly(ethyleneglycol) or a derivative thereof, and especially with a molecular weight in the range from about 25000 Da to about 40000 Da.

Each polymer molecule attached to the modified antibody fragment may be covalently linked to the sulphur atom of a cysteine residue located in the fragment. The covalent linkage will generally be a disulphide bond or, in particular, a sulphur-carbon bond.

Where desired, the antibody fragment may have one or more effector or reporter molecules attached to it. The effector or reporter molecules may be attached to the antibody fragment through any available amino acid side-chain or terminal amino acid functional group located in the fragment, for example any free amino, imino, hydroxyl or carboxyl group.

An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar Therapeutics, Inc (Huntsville, Ala.) or may be prepared from commercially available starting materials using conventional chemical procedures.

Standard chemical or recombinant DNA procedures in which the antibody fragment is linked either directly or via a coupling agent to the effector or reporter molecule either before or after reaction with the activated polymer as appropriate may be used. Particular chemical procedures include, for example, those described in WO 93/06231, WO 92/22583, WO 90/09195, WO 89/01476, WO 99/15549 and WO03/031581. Alternatively, where the effector or reporter molecule is a protein or polypeptide the linkage may be achieved using recombinant DNA procedures, for example as described in WO 86/01533 and EP 0392745. Most preferably antibodies are attached to poly(ethyleneglycol) (PEG) moieties. Preferably, a modified Fab fragment is PEGylated, i.e. has PEG (poly(ethyleneglycol)) covalently attached thereto, e.g. according to the method disclosed in EP 0948544 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one embodiment, a PEG modified Fab fragment has a maleimide group covalently linked to a single thiol group in a modified hinge region. A lysine residue may be covalently linked to the maleimide group. To each of the amine groups on the lysine residue may be attached a methoxypoly(ethyleneglycol) polymer having a molecular weight of approximately 20,000 Da. The total molecular weight of the entire effector molecule may therefore be approximately 40,000 Da.

KIAA0659 polypeptides or cells expressing said polypeptides can be used to produce antibodies, e.g. which specifically recognise said KIAA0659 polypeptides. Antibodies generated against a KIAA0659 polypeptide may be obtained by administering the polypeptides to an animal, preferably a non-human animal, using well-known and routine protocols.

Anti-KIAA0659 antibodies include functionally active fragments, derivatives or analogues and may be, but are not limited to, polyclonal, monoclonal, bi-, tri- or tetra-valent antibodies, humanized or chimeric antibodies, single chain antibodies, Fab fragments, Fab′ and Fab′₂ fragments, fragments produced by a Fab expression library, anti-idiotypic (anti-Id) antibodies, and epitope-binding fragments of any of the above. Humanized antibodies are antibody molecules from non-human species having one or more complementarity determining regions (CDRs) from the non-human species and a framework region from a human immunoglobulin molecule (see, e.g. U.S. Pat. No. 5,585,089). Antibodies include immunoglobulin molecules and immunologically active portions of immunoglobulin molecules, i.e. molecules that contain an antigen binding site that specifically binds an antigen. The immunoglobulin molecules of the invention can be of any class (e.g. IgG, IgE, IgM, IgD and IgA) or subclass of immunoglobulin molecule.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Chimeric antibodies are those antibodies encoded by immunoglobulin genes that have been genetically engineered so that the light and heavy chain genes are composed of immunoglobulin gene segments belonging to different species. These chimeric antibodies are likely to be less antigenic. Bivalent antibodies may be made by methods known in the art (Milstein et al., 1983, Nature 305:537-539; WO 93/08829, Traunecker et al., 1991, EMBO J. 10:3655-3659). Bi-, tri- and tetra-valent antibodies may comprise multiple specificities or may be monospecific (see for example WO 92/22853).

The antibodies for use in the invention may be generated using single lymphocyte antibody methods based on the molecular cloning and expression of immunoglobulin variable region cDNAs generated from single lymphocytes that were selected for the production of specific antibodies such as described by Babcook, J. et al., 1996, Proc. Natl. Acad. Sci. USA 93(15):7843-7848 and in WO92/02551.

The antibodies for use in the present invention can also be generated using various phage display methods known in the art and include those disclosed by Brinkman et al. (in J. Immunol. Methods, 1995, 182:41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. Techniques for the production of single chain antibodies, such as those described in U.S. Pat. No. 4,946,778 can also be adapted to produce single chain antibodies to KIAA0659 polypeptides. Also, transgenic mice, or other organisms, including other mammals, may be used to express humanized antibodies.

KIAA0659 polypeptides can be used for the identification of agents for use in the methods of treatment and/or prophylaxis according to the invention.

A further aspect of the invention provides methods of screening for anti-carcinoma agents that interact with a KIAA0659 polypeptide comprising:
(a) contacting said polypeptide with a candidate agent; and
(b) determining whether or not the candidate agent interacts with said polypeptide.

Preferably, the determination of an interaction between the candidate agent and KIAA0659 polypeptide comprises quantitatively detecting binding of the candidate agent and said polypeptide.

Further provided is a method of screening for anti-carcinoma agents that modulate the expression or activity of a KIAA0659 polypeptide comprising:
(i) comparing the expression or activity of said polypeptide in the presence of a candidate agent with the expression or activity of said polypeptide in the absence of the candidate agent or in the presence of a control agent; and
(ii) determining whether the candidate agent causes the expression or activity of said polypeptide to change.

Preferably, the expression and/or activity of a KIAA0659 polypeptide is compared with a predetermined reference range or control.

More preferably the method further comprises selecting an agent, which interacts with a KIAA0659 polypeptide or is capable of modulating the interaction, expression or activity of a KIAA0659 polypeptide, for further testing for use in the treatment and/or prophylaxis of carcinoma. It will be apparent to one skilled in the art that the above screening methods are also appropriate for screening for anti-carcinoma agents which interact with or modulate the expression or activity of a KIAA0659 nucleic acid molecule.

The invention also provides assays for use in drug discovery in order to identify or verify the efficacy of agents for treatment and/or prophylaxis of carcinoma. Agents identified using these methods can be used as lead agents for drug discovery, or used therapeutically. Expression of a KIAA0659 polypeptide can be assayed by, for example, immunoassays, gel electrophoresis followed by visualisation, detection of mRNA or KIAA0659 polypeptide activity, or any other method taught herein or known to those skilled in the art. Such assays can be used to screen candidate agents, in clinical monitoring or in drug development.

Agents can be selected from a wide variety of candidate agents. Examples of candidate agents include but are not limited to, nucleic acids (e.g. DNA and RNA), carbohydrates, lipids, proteins, polypeptides, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is suited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Examples of suitable methods based on the present description for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, for example, in solution (e.g. Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et a., 1990, Proc. Natl. Acad. Sci. USA 87:6378-6382; and Felici, 1991, J. Mol. Biol. 222:301-310).

In one embodiment, agents that interact with (e.g. bind to) a KIAA0659 polypeptide are identified in a cell-based assay where a population of cells expressing a KIAA0659 polypeptide is contacted with a candidate agent and the ability of the candidate agent to interact with the polypeptide is determined. Preferably, the ability of a candidate agent to interact with a KIAA0659 polypeptide is compared to a reference range or control. In another embodiment, a first and second population of cells expressing a KIAA0659 polypeptide are contacted with a candidate agent or a control agent and the ability of the candidate agent to interact with the polypeptide is determined by comparing the difference in interaction between the candidate agent and control agent. If desired, this type of assay may be used to screen a plurality (e.g. a library) of candidate agents using a plurality of cell populations expressing a KIAA0659 polypeptide. If desired, this assay may be used to screen a plurality (e.g. a library) of candidate agents. The cell, for example, can be of prokaryotic origin (e.g. E. coli) or eukaryotic origin (e.g. yeast or mammalian). Further, the cells can express the KIAA0659 polypeptide endogenously or be genetically engineered to express the polypeptide. In some embodiments, a KIAA0659 polypeptide or the candidate agent is labelled, for example with a radioactive label (such as ³²P, ³⁵S or ¹²⁵I or a fluorescent label (such as fluorescein isothiocyanate, rhodamine, phycoerythrin, phycocyanin, allophycocyanin, o-phthaldehyde or fluorescamine) to enable detection of an interaction between a polypeptide and a candidate agent.

In another embodiment, agents that interact with (e.g. bind to) a KIAA0659 polypeptide are identified in a cell-free assay system where a sample expressing a KIAA0659 polypeptide is contacted with a candidate agent and the ability of the candidate agent to interact with the polypeptide is determined. Preferably, the ability of a candidate agent to interact with a KIAA0659 polypeptide is compared to a reference range or control. In a preferred embodiment, a first and second sample comprising native or recombinant KIAA0659 polypeptide are contacted with a candidate agent or a control agent and the ability of the candidate agent to interact with the polypeptide is determined by comparing the difference in interaction between the candidate agent and control agent. If desired, this assay may be used to screen a plurality (e.g. a library) of candidate agents using a plurality of KIAA0659 polypeptide samples. Preferably, the polypeptide is first immobilized, by, for example, contacting the polypeptide with an immobilized antibody which specifically recognizes and binds it, or by contacting a purified preparation of polypeptide with a surface designed to bind proteins. The polypeptide may be partially or completely purified (e.g. partially or completely free of other polypeptides) or part of a cell lysate. Further, the polypeptide may be a fusion protein comprising the KIAA0659 polypeptide or a biologically active portion thereof and a domain such as glutathionine-S-transferase. Alternatively, the polypeptide can be biotinylated using techniques well known to those of skill in the art (e.g. biotinylation kit, Pierce Chemicals; Rockford, Ill.). The ability of the candidate agent to interact with the polypeptide can be duplicated by methods known to those of skill in the art.

In one embodiment, a KIAA0659 polypeptide is used as a “bait protein” in a two-hybrid assay or three hybrid assay to identify other proteins that bind to or interact with the KIAA0659 polypeptide (see e.g. U.S. Pat. No. 5,283,317; Zervos et al., 1993, Cell 72:223-232; Madura et al. 1993, J. Biol. Chem. 268:12046-12054; Bartel et al., 1993, Bio/Techniques 14:920-924; Iwabuchi et al., 1993, Oncogene 8:1693-1696; and WO 94/10300). As those skilled in the art will appreciate, such binding proteins are also likely to be involved in the propagation of signals by a KIAA0659 polypeptide. For example, they may be upstream or downstream elements of a signalling pathway involving a KIAA0659 polypeptide. Alternatively, polypeptides that interact with a KIAA0659 polypeptide can be identified by isolating a protein complex comprising a KIAA0659 polypeptide (said polypeptide may interact directly or indirectly with one or more other polypeptides) and identifying the associated proteins using methods known in the art such as mass spectrometry or Western blotting (for examples see Blackstock, W. & Weir, M. 1999, Trends in Biotechnology, 17:121-127; Rigaut, G. 1999, Nature Biotechnology, 17:1030-1032; Husi, H. 2000, Nature Neurosci. 3:661-669; Ho, Y. et al., 2002, Nature, 415:180-183; Gavin, A. et al., 2002, Nature, 415:141-147).

In all cases, the ability of the candidate agent to interact directly or indirectly with the KIAA0659 polypeptide can be determined by methods known to those of skill in the art. For example but without limitation, the interaction between a candidate agent and a KIAA0659 polypeptide can be determined by flow cytometry, a scintillation assay, an activity assay, mass spectrometry, microscopy, immunoprecipitation or western blot analysis.

In yet another embodiment, agents that competitively interact with (i.e. competitively binding to) a KIAA0659 polypeptide are identified in a competitive binding assay and the ability of the candidate agent to interact with the KIAA0659 polypeptide is determined. Preferably, the ability of a candidate agent to interact with a KIAA0659 polypeptide is compared to a reference range or control. In a preferred embodiment, a first and second population of cells expressing both a KIAA0659 polypeptide and a protein which is known to interact with the KIAA0659 polypeptide are contacted with a candidate agent or a control agent. The ability of the candidate agent to competitively interact with the KIAA0659 polypeptide is then determined by comparing the interaction in the first and second population of cells. In another embodiment, an alternative second population or a further population of cells may be contacted with an agent which is known to competitively interact with a KIAA0659 polypeptide. Alternatively, agents that competitively interact with a KIAA0659 polypeptide are identified in a cell-free assay system by contacting a first and second sample comprising a KIAA0659 polypeptide and a protein known to interact with the KIAA0659 polypeptide with a candidate agent or a control agent. The ability of the candidate agent to competitively interact with the KIAA0659 polypeptide is then determined by comparing the interaction in the first and second sample. In another embodiment, an alternative second sample or a further sample comprising a KIAA0659 polypeptide may be contacted with an agent which is known to competitively interact with a KIAA0659 polypeptide. In any case, the KIAA0659 polypeptide and known interacting protein may be expressed naturally or may be recombinantly expressed; the candidate agent may be added exogenously, or be expressed naturally or recombinantly.

In another embodiment, agents that modulate the interaction between a KIAA0659 polypeptide and another agent, for example but without limitation a protein, may be identified in a cell-based assay by contacting cells expressing a KIAA0659 polypeptide in the presence of a known interacting agent and a candidate modulating agent and selecting the candidate agent which modulates the interaction. Alternatively, agents that modulate an interaction between a KIAA0659 polypeptide and another agent, for example but without limitation a protein, may be identified in a cell-free assay system by contacting the polypeptide with an agent known to interact with the polypeptide in the presence of a candidate agent. A modulating agent can act as an antibody, a cofactor, an inhibitor, an activator or have an antagonistic or agonistic effect on the interaction between a KIAA0659 polypeptide and a known agent. As stated above the ability of the known agent to interact with a KIAA0659 polypeptide can be determined by methods known in the art. These assays, whether cell-based or cell-free, can be used to screen a plurality (e.g. a library) of candidate agents.

In another embodiment, a cell-based assay system is used to identify agents capable of modulating (i.e. stimulating or inhibiting) the activity of a KIAA0659 polypeptide. Accordingly, the activity of a KIAA0659 polypeptide is measured in a population of cells that naturally or recombinantly express a KIAA0659 polypeptide, in the presence of a candidate agent. Preferably, the activity of a KIAA0659 polypeptide is compared to a reference range or control. In a preferred embodiment, the activity of a KIAA0659 polypeptide is measured in a first and second population of cells that naturally or recombinantly express a KIAA0659 polypeptide, in the presence of agent or absence of a candidate agent (e.g. in the presence of a control agent) and the activity of the KIAA0659 polypeptide is compared. The candidate agent can then be identified as a modulator of the activity of a KIAA0659 polypeptide based on this comparison. Alternatively, the activity of a KIAA0659 polypeptide can be measured in a cell-free assay system where the KIAA0659 polypeptide is either natural or recombinant. Preferably, the activity of a KIAA0659 polypeptide is compared to a reference range or control. In a preferred embodiment, the activity of a KIAA0659 polypeptide is measured in a first and second sample in the presence or absence of a candidate agent and the activity of the KIAA0659 polypeptide is compared. The candidate agent can then be identified as a modulator of the activity of a KIAA0659 polypeptide based on this comparison.

The activity of a KIAA0659 polypeptide can be assessed by detecting its effect on a downstream effector, for example but without limitation, the level or activity of a second messenger (e.g. cAMP, intracellular Ca²⁺, diacylglycerol, IP₃, etc.), detecting catalytic or enzymatic activity, detecting the induction of a reporter gene (e.g. luciferase) or detecting a cellular response, for example, proliferation, differentiation or transformation where appropriate as known by those skilled in the art (for activity measurement techniques see, e.g. U.S. Pat. No. 5,401,639). The candidate agent can then be identified as a modulator of the activity of a KIAA0659 polypeptide by comparing the effects of the candidate agent to the control agent. Suitable control agents include PBS or normal saline.

In another embodiment, agents such as an enzyme, or a biologically active portion thereof, which is responsible for the production or degradation of a KIAA0659 polypeptide or is responsible for the post-translational modification of a KIAA0659 polypeptide can be identified. In a primary screen, substantially pure, native or recombinantly expressed KIAA0659 polypeptides, nucleic acids or cellular extract or other sample comprising native or recombinantly expressed KIAA0659 polypeptides or nucleic acids are contacted with a plurality of candidate agents (for example but without limitation, a plurality of agents presented as a library) that may be responsible for the processing of a KIAA0659 polypeptide or nucleic acid, in order to identify such agents. The ability of the candidate agent to modulate the production, degradation or post-translational modification of a KIAA0659 polypeptide or nucleic acid can be determined by methods known to those of skill in the art, including without limitation, flow cytometry, radiolabelling, a kinase assay, a phosphatase assay, immunoprecipitation and Western blot analysis, or Northern blot analysis.

In yet another embodiment, cells expressing a KIAA0659 polypeptide are contacted with a plurality of candidate agents. The ability of such an agent to modulate the production, degradation or post-translational modification of a KIAA0659 polypeptide can be determined by methods known to those of skill in the art, as described above.

In one embodiment, agents that modulate the expression of a KIAA0659 polypeptide (e.g. down-regulate) are identified in a cell-based assay system. Accordingly, a population of cells expressing a KIAA0659 polypeptide or nucleic acid are contacted with a candidate agent and the ability of the candidate agent to alter expression of the KIAA0659 polypeptide or nucleic acid is determined by comparison to a reference range or control. In another embodiment, a first and second population of cells expressing a KIAA0659 polypeptide are contacted with a candidate agent or a control agent and the ability of the candidate agent to alter the expression of the KIAA0659 polypeptide or nucleic acid is determined by comparing the difference in the level of expression of the KIAA0659 polypeptide or nucleic acid between the first and second populations of cells. In a further embodiment, the expression of the KIAA0659 polypeptide or nucleic acid in the first population may be further compared to a reference range or control. If desired, this assay may be used to screen a plurality (e.g. a library) of candidate agents. The cell, for example, can be of prokaryotic origin (e.g. E. coli) or eukaryotic origin (e.g. yeast or mammalian). Further, the cells can express a KIAA0659 polypeptide or nucleic acid endogenously or be genetically engineered to express a KIAA0659 polypeptide or nucleic acid. The ability of the candidate agents to alter the expression of a KIAA0659 polypeptide or nucleic acid can be determined by methods known to those of skill in the art, for example and without limitation, by flow cytometry, radiolabelling, a scintillation assay, immunoprecipitation, Western blot analysis or Northern blot analysis.

In another embodiment, agents that modulate the expression of a KIAA0659 polypeptide or nucleic acid are identified in an animal model. Examples of suitable animals include, but are not limited to, mice, rats, rabbits, monkeys, guinea pigs, dogs and cats. Preferably, the animal used represents a model of carcinoma. Accordingly, a first and second group of mammals are administered with a candidate agent or a control agent and the ability of the candidate agent to modulate the expression of the KIAA0659 polypeptide or nucleic acid is determined by comparing the difference in the level of expression between the first and second group of mammals. Where desired, the expression levels of the KIAA0659 polypeptides or nucleic acid in the first and second groups of mammals can be compared to the level of a KIAA0659 polypeptide or nucleic acid in a control group of mammals. The candidate agent or a control agent can be administered by means known in the art (e.g. orally, rectally or parenterally such as intraperitoneally or intravenously). Changes in the expression of a polypeptide or nucleic acid can be assessed by the methods outlined above. In a particular embodiment, a therapeutically effective amount of an agent can be determined by monitoring an amelioration or improvement in disease symptoms, to delay onset or slow progression of the disease, for example but without limitation, a reduction in tumour size. Techniques known to physicians familiar with carcinoma can be used to determine whether a candidate agent has altered one or more symptoms associated with the disease.

One skilled in the art will also appreciate that a KIAA0659 polypeptide may also be used in a method for the structure-based design of an agent, in particular a small molecule which acts to modulate (e.g. stimulate or inhibit) the activity of said polypeptide, said method comprising:
1) determining the three-dimensional structure of said polypeptide;
2) deducing the three-dimensional structure within the polypeptide of the likely reactive or binding site(s) of the agent;
3) synthesising candidate agents that are predicted to react or bind to the deduced reactive or binding site; and
4) testing whether the candidate agent is able to modulate the activity of said polypeptide.

It will be appreciated that the method described above is likely to be an iterative process.

As discussed herein, agents which interact with a KIAA0659 polypeptide find use in the treatment and/or prophylaxis of carcinoma. For such use the agents will generally be administered in the form of a pharmaceutical composition.

Thus, according to the invention there is provided a pharmaceutical composition comprising an agent which interacts with a KIAA0659 polypeptide and a pharmaceutically acceptable diluent, excipient and/or carrier. Pharmaceutical compositions may also find use as a vaccine and may comprise additional components acceptable for vaccine use and may additionally comprise one or more suitable adjuvants as known to the skilled person.

Hereinafter, the agents of use in the invention, KIAA0659 polypeptides and KIAA0659 nucleic acids of use in treatment and/or prophylaxis are referred to as ‘active agents’. When a reference is made herein to a method of treating or preventing a disease or condition using a particular active agent or combination of agents, it is to be understood that such a reference is intended to include the use of that active agent or combination of agents in the preparation of a medicament for the treatment and/or prophylaxis of the disease or condition.

The composition will usually be supplied as part of a sterile, pharmaceutical composition that will normally include a pharmaceutically acceptable carrier. This composition may be in any suitable form (depending upon the desired method of administering it to a patient).

Active agents of the invention may be administered to a subject by any of the routes conventionally used for drug administration, for example they may be administered parenterally, orally, topically (including buccal, sublingual or transdermal) or by inhalation. The most suitable route for administration in any given case will depend on the particular active agent, the carcinoma involved, the subject, and the nature and severity of the disease and the physical condition of the subject.

The active agents may be administered in combination, e.g. simultaneously, sequentially or separately, with one or more other therapeutically active, e.g. anti-tumour, compounds.

Pharmaceutical compositions may be conveniently presented in unit dose forms containing a predetermined amount of an active agent of the invention per dose. Such a unit may contain for example but without limitation, 750 mg/kg to 0.1 mg/kg depending on the condition being treated, the route of administration and the age, weight and condition of the subject.

Pharmaceutically acceptable carriers for use in the invention may take a wide variety of forms depending, e.g. on the route of administration.

Compositions for oral administration may be liquid or solid. Oral liquid preparations may be in the form of, for example, aqueous or oily suspensions, solutions, emulsions, syrups or elixirs, or may be presented as a dry product for reconstitution with water or other suitable vehicle before use. Oral liquid preparations may contain suspending agents as known in the art.

In the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which case solid pharmaceutical carriers are generally employed. In addition to the common dosage forms set out above, active agents of the invention may also be administered by controlled release means and/or delivery devices. Tablets and capsules may comprise conventional carriers or excipients such as binding agents for example, syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated by standard aqueous or non-aqueous techniques according to methods well known in normal pharmaceutical practice.

Pharmaceutical compositions of the present invention suitable for oral administration may be presented as discrete units such as capsules, cachets or tablets, each containing a predetermined amount of the active agent, as a powder or granules, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active agent with the carrier, which constitutes one or more necessary ingredients. In general, the compositions are prepared by uniformly and intimately admixing the active agent with liquid carriers or finely divided solid carriers or both, and then, if necessary, shaping the product into the desired presentation. For example, a tablet may be prepared by compression or moulding, optionally with one or more accessory ingredients.

Pharmaceutical compositions suitable for parenteral administration may be prepared as solutions or suspensions of the active agents of the invention in water suitably mixed with a surfactant such as hydroxypropylcellulose. Dispersions can also be prepared in glycerol, liquid polyethylene glycols, and mixtures thereof in oils. Under ordinary conditions of storage and use, these preparations contain a preservative to prevent the growth of microorganisms.

The pharmaceutical forms suitable for injectable use include aqueous or non-aqueous sterile injection solutions which may contain anti-oxidants, buffers, bacteriostats and solutes which render the composition isotonic with the blood of the intended recipient, and aqueous and non-aqueous sterile suspensions which may include suspending agents and thickening agents. Extemporaneous injection solutions, dispersions and suspensions may be prepared from sterile powders, granules and tablets.

Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the pharmaceutical compositions of the invention can be formulated to ensure proper distribution in vivo. For example, the blood-brain barrier excludes many highly hydrophilic compounds and it may be preferable to deliver pharmaceutical compositions in liposomes. Thus, in one embodiment of the invention, the active agents of the invention are formulated in liposomes; in a more preferred embodiment, the liposomes include a targeting moiety. In a most preferred embodiment, the therapeutic compounds in the liposomes are delivered by bolus injection to a site proximal to the tumour. For methods of manufacturing liposomes, see, e.g. U.S. Pat. No. 4,522,811; 5,374,548; and 5,399,331. The liposomes may comprise one or more moieties which are selectively transported into specific cells or organs, thus enhancing targeted drug delivery (see, e.g. Ranade, W. 1989, J. Clin. Pharmacol. 29:685). Exemplary targeting moieties include folate or biotin (see, e.g. U.S. Pat. No. 5,416,016.); mannosides (Umezawa et al., 1988, Biochem. Biophys. Res. Commun. 153:1038); antibodies (Bloeman, PG. et al., 1995, FEBS Lett. 357:140; M. Owais et al., 1995, Antimicrob. Agents Chemother. 39:180); surfactant protein A receptor (Briscoe et al., 1995, Am. J. Physiol. 1233:134), different species of which may comprise the formulations of the inventions, as well as components of the invented molecules; p120 (Schreier et al., 1994, J. Biol. Chem. 269:9090); see also Keinanen, K. & Laukkanen, M L. 1994, FEBS Lett. 346:123; Killion, J J. & Fidler, I J. 1994, Immunomethods 4:273. The compositions may be presented in unit-dose or multi-dose containers, for example in sealed ampoules and vials and to enhance stability, may be stored in a freeze-dried (lyophilized) condition requiring only the addition of the sterile liquid carrier, for example water for injections, immediately prior to use. The sterile liquid carrier may be supplied in a separate vial or ampoule and can be a solvent or dispersion medium containing, for example, water, ethanol, polyol (e.g. glycerol, propylene glycol and liquid polyethylene glycol), suitable mixtures thereof, and vegetable oils. Advantageously, agents such as a local anaesthetic, preservative and buffering agents can be included the sterile liquid carrier.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollients in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base.

Pharmaceutical compositions adapted for topical administration in the mouth include lozenges, pastilles and mouth washes.

Pharmaceutical compositions adapted for topical administration to the eye include eye drops wherein the active agent is dissolved or suspended in a suitable carrier, especially an aqueous solvent. They also include topical ointments or creams as above.

Pharmaceutical compositions suitable for rectal administration wherein the carrier is a solid are most preferably presented as unit dose suppositories. Suitable carriers include cocoa butter or other glyceride or materials commonly used in the art, and the suppositories may be conveniently formed by admixture of the combination with the softened or melted carrier(s) followed by chilling and shaping moulds. They may also be administered as enemas.

Pharmaceutical compositions adapted for vaginal administration may be presented as pessaries, tampons, creams, gels, pastes, foams or spray compositions. These may comprise emollients or bases as commonly used in the art.

The dosage to be administered of an active agent will vary according to the particular active agent, the carcinoma involved, the subject, and the nature and severity of the disease and the physical condition of the subject, and the selected route of administration; the appropriate dosage can be readily determined by a person skilled in the art. For the treatment and/or prophylaxis of carcinoma in humans and animals pharmaceutical compositions comprising antibodies can be administered to patients (e.g., human subjects) at therapeutically or prophylactically effective dosages (e.g. dosages which result in tumour growth inhibition and/or tumour cell migration inhibition) using any suitable route of administration, such as injection and other routes of administration known in the art for antibody-based clinical products.

The compositions may contain from 0.1% by weight, preferably from 10-60%, or more, by weight, of the active agent of the invention, depending on the method of administration.

It will be recognized by one of skill in the art that the optimal quantity and spacing of individual dosages of an active agent of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

KIAA0659 polypeptides may also be of use in the treatment and/or prophylaxis of carcinoma. Accordingly, provided is a method for the treatment and/or prophylaxis of carcinoma comprising administering a therapeutically effective amount of a composition comprising a KIAA0659 polypeptide, preferably as a vaccine. Also provided is the use of a KIAA0659 polypeptide for the manufacture of a medicament for the treatment and/or prophylaxis of carcinoma. Where they are provided for use with the methods of the invention KIAA0659 are preferably provided in isolated form. More preferably the KIAA0659 polypeptides have been purified to at least some extent. KIAA0659 polypeptides can also be produced using recombinant methods, synthetically produced or produced by a combination of these methods. KIAA0659 polypeptides may be provided in substantially pure form, that is to say free, to a substantial extent, from other proteins.

Recombinant KIAA0659 polypeptides may be prepared by processes well known in the art from genetically engineered host cells comprising expression systems. Accordingly, the present invention also relates to expression systems which comprise a KIAA0659 polypeptide or KIAA0659 nucleic acid, to host cells which are genetically engineered with such expression systems and to the production of KIAA0659 polypeptides by recombinant techniques. Cell-free translation systems can also be employed to produce recombinant polypeptides (e.g. rabbit reticulocyte lysate, wheat germ lysate, SP6/T7 in vitro T&T and RTS 100 E. Coli HY transcription and translation kits from Roche Diagnostics Ltd., Lewes, UK and the TNT Quick coupled Transcription/Translation System from Promega UK, Southampton, UK.

For recombinant KIAA0659 polypeptide production, host cells can be genetically engineered to incorporate expression systems or portions thereof for KIAA0659 nucleic acids. Such incorporation can be performed using methods well known in the art, such as, calcium phosphate transfection, DEAD-dextran mediated transfection, transvection, microinjection, cationic lipid-mediated transfection, electroporation, transduction, scrape loading, ballistic introduction or infection (see e.g. Davis et al., Basic Methods in Molecular Biology, 1986 and Sambrook et al., Molecular Cloning: A Laboratory Manual, 2^{nd} Ed., Cold Spring Harbour laboratory Press, Cold Spring Harbour, N.Y., 1989).

Representative examples of host cells include bacterial cells e.g. E. Coli, Streptococci, Staphylococci, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, HEK 293, BHK and Bowes melanoma cells; and plant cells.

A wide variety of expression systems can be used, such as and without limitation, chromosomal, episomal and virus-derived systems, e.g. vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a nucleic acid to produce a polypeptide in a host may be used. The appropriate nucleic acid sequence may be inserted into an expression system by any variety of well-known and routine techniques, such as those set forth in Sambrook et al., supra. Appropriate secretion signals may be incorporated into the KIAA0659 polypeptide to allow secretion of the translated protein into the lumen of the endoplasmic reticulum, the periplasmic space or the extracellular environment. These signals may be endogenous to the KIAA0659 polypeptide or they may be heterologous signals.

If a KIAA0659 polypeptide is to be expressed for use in cell-based screening assays, it is preferred that the polypeptide be produced at the cell surface. In this event, the cells may be harvested prior to use in the screening assay. If the KIAA0659 polypeptide is secreted into the medium, the medium can be recovered in order to isolate said polypeptide. If produced intracellularly, the cells must first be lysed before the KIAA0659 polypeptide is recovered.

KIAA0659 polypeptides can be recovered and purified from recombinant cell cultures or from other biological sources by well-known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, an antibody which specifically binds to a KIAA0659 polypeptide can be used to deplete a sample comprising a KIAA0659 polypeptide of said polypeptide or to purify said polypeptide. Techniques well-known in the art, may be used for refolding to regenerate native or active conformations of the KIAA0659 polypeptides when the polypeptides have been denatured during isolation and or purification. In the context of the present invention, KIAA0659 polypeptides can be obtained from a biological sample from any source, such as and without limitation, a lung or liver tissue sample.

KIAA0659 polypeptides may be in the form of a ‘mature’ protein or may be part of a larger protein such as a fusion protein. It is often advantageous to include an additional amino acid sequence which contains secretory or leader sequences, a pre-, pro- or prepro-protein sequence, or a sequence which aids in purification such as an affinity tag, for example, but without limitation, multiple histidine residues, a FLAG tag, HA tag or myc tag. An additional sequence which may provide stability during recombinant production may also be used. Such sequences may be optionally removed as required by incorporating a cleavable sequence as an additional sequence or part thereof. Thus, a KIAA0659 polypeptide may be fused to other moieties including other polypeptides. Such additional sequences and affinity tags are well known in the art.

Amino acid substitutions may be conservative or semi-conservative as known in the art and preferably do not significantly affect the desired activity of the polypeptide. Substitutions may be naturally occurring or may be introduced for example using mutagenesis (e.g. Hutchinson et al., 1978, J. Biol. Chem. 253:6551). Thus, the amino acids glycine, alanine, valine, leucine and isoleucine can often be substituted for one another (amino acids having aliphatic side chains). Of these possible substitutions, it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains);
cysteine and methionine (amino acids having sulphur-containing side chains); and
aspartic acid and glutamic acid can substitute for phospho-serine and phospho-threonine, respectively (amino acids with acidic side chains).

In one particular embodiment, the substituted amino acid(s) do significantly affect the activity of the KIAA0659 polypeptide and may be selected specifically to render dominant negative activity upon the peptide. In another embodiment, the substituted amino acid(s) may be selected specifically to render the polypeptide constitutively active.

Modifications include naturally occurring modifications such as and without limitation, post-translational modifications and also non-naturally occurring modifications such as may be introduced by mutagenesis.

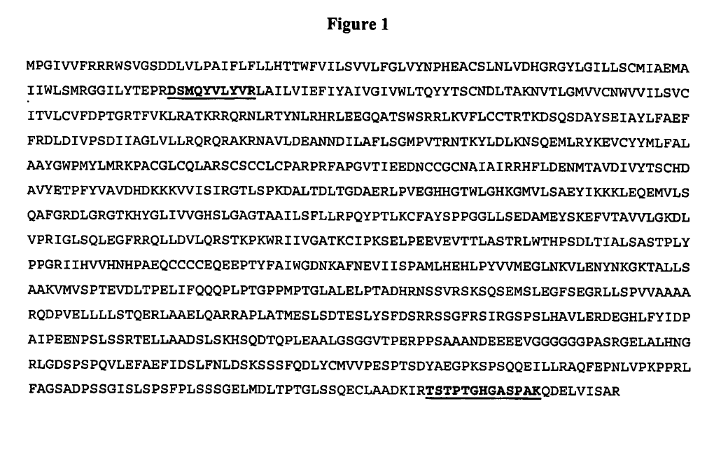

Preferably a derivative of a KIAA0659 polypeptide has at least 70% identity to an amino acid sequence shown in FIG. 1 (SEQ ID NO: 1), more preferably it has at least 75%, at least 80%, at least 85%, at least 90%, at least 95% or at least 98% identity. Percentage identity is a well known concept in the art and can be calculated using, for example but without limitation, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656).

A fragment of a KIAA0659 polypeptide may also be of use in the methods of the invention and includes a fragment of a polypeptide having the amino acid sequence of SEQ ID NO: 1, which has at least 70% homology over the length of the fragment. Preferably, said fragments are at least 10 amino acids in length, preferably they are at least 20, at least 30, at least 50 or at least 100 amino acids in length. A fragment has at least 70% identity over its length to an amino acid sequence shown in FIG. 1 (SEQ ID NO:1), more preferably it has at least 75%, at least 80%, at least 85%, at least 90%, at least 95% or at least 98% identity.

Where a KIAA0659 polypeptide is the active agent of a pharmaceutical composition for use in the treatment and/or prophylaxis of carcinoma, preferably recombinant KIAA0659 polypeptides are used. In a particular embodiment, a KIAA0659 polypeptide fused to another polypeptide, such as the protein transduction domain of the HIV/Tat protein which facilitates the entry of the fusion protein into a cell (Asoh, S. et al., 2002, Proc. Natl. Acad. Sci. USA, 99:17107-17112), is provided for use in the manufacture of a medicament for the treatment and/or prophylaxis of carcinoma.

In another aspect, detection of a KIAA0659 polypeptide in a subject with carcinoma may be used to identify in particular an appropriate patient population for treatment according to the methods of the invention.

Accordingly, the present invention provides a method of screening for and/or diagnosis or prognosis of carcinoma in a subject, and/or monitoring the effectiveness of carcinoma therapy, which comprises the step of detecting and/or quantifying in a biological sample obtained from said subject a KIAA0659 polypeptide. The KIAA0659 polypeptide for use in the method of screening and/or diagnosis preferably:
(a) comprises or consists of the amino acid sequence of SEQ ID NO:1;
(b) is a derivative having one or more amino acid substitutions, modifications, deletions or insertions relative to the amino acid sequence of SEQ ID NO:1 which retains the activity of KIAA0659; or
(c) is a fragment of a polypeptide having the amino acid sequence of SEQ ID NO:1, which is at least ten amino acids long and has at least 70% homology over the length of the fragment.

In one aspect, the expression is compared to a previously determined reference range. Preferably, the step of detecting comprises:
(a) contacting the sample with a capture reagent that is specific for a polypeptide as defined in (a) to (c), above; and
(b) detecting whether binding has occurred between the capture reagent and said polypeptide in the sample.

In another aspect, the captured polypeptide is detected using a directly or indirectly labelled detection reagent which may be immobilised on a solid phase.

A convenient means for detecting/quantifying a KIAA0659 polypeptide involves the use of antibodies. A KIAA0659 polypeptide can be used as an immunogen to raise antibodies which interact with (bind to or recognise) said polypeptide using methods known in the art as described above. Thus, in a further aspect, the present invention provides the use of an antibody that specifically binds to at least one KIAA0659 polypeptide for screening for and/or diagnosis of carcinoma in a subject or for monitoring the efficacy of an anti-carcinoma therapy. In a particular embodiment, the methods of diagnosis using an anti-KIAA0659 polypeptide antibody can be used to identify an appropriate patient population for treatment according to the methods of the invention.

KIAA0659 antibodies can also be used, inter alia, for the diagnosis of carcinoma by detecting KIAA0659 expression in a biological sample of human tissue and/or in subfractions thereof, for example but without limitation, membrane, cytosolic or nuclear subfractions.

In a further aspect, the method of detecting a KIAA0659 polypeptide in a biological sample comprises detecting and/or quantitating the amount of the KIAA0659 polypeptide in said sample using a directly or indirectly labelled detection reagent. A KIAA0659 polypeptide can be detected by means of any immunoassay known in the art, including, without limitation, immunoprecipitation followed by sodium dodecyl sulfate polyacrylamide gel electrophoresis, 2 dimensional gel electrophoresis, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

Detection of the interaction of an antibody with an antigen can be facilitated by coupling the antibody to a detectable substance for example, but without limitation, an enzyme (such as horseradish peroxidase, alkaline phosphatase, beta-galactosidase, acetylcholinesterase), a prosthetic group (such as streptavidin, avidin, biotin), a fluorescent material (such as umbelliferone, fluorescein, fluorescein isothiocyanate, rhodamine, dichlorotriazinylamine fluorescein, dansyl chloride, phycoerythrin), a luminescent material (such as luminol), a bioluminescent material (such as luciferase, luciferin, aequorin), a radioactive nuclide (such as ¹²⁵I, ¹³¹I, ¹¹¹In, ⁹⁹Tc) a positron emitting metal or a non-radioactive paramagnetic metal ion (see U.S. Pat. No. 4,741,900).

The invention also provides diagnostic kits, comprising a capture reagent (e.g. an antibody) against a KIAA0659 polypeptide as defined above. In addition, such a kit may optionally comprise one or more of the following:
(1) instructions for using the capture reagent for screening, diagnosis, prognosis, therapeutic monitoring or any combination of these applications;
(2) a labelled binding partner to the capture reagent;
(3) a solid phase (such as a reagent strip) upon which the capture reagent is immobilised; and
(4) a label or insert indicating regulatory approval for screening, diagnostic, prognostic or therapeutic use or any combination thereof.

If no labelled binding partner to the capture reagent is provided, the anti-KIAA0659 polypeptide capture reagent itself can be labelled with a detectable marker, e.g. a chemiluminescent, enzymatic, fluorescent, or radioactive moiety (see above).

It will also be apparent to one skilled in the art that detection and/or quantitation of a KIAA0659 nucleic acid may be used in a method of screening for and/or diagnosis or prognosis of carcinoma in a subject, and/or monitoring the effectiveness of carcinoma therapy.

Unless the context indicates otherwise, KIAA0659 nucleic acids include those nucleic acid molecules which may have one or more of the following characteristics and thus may:
d) comprise or consist of the DNA sequence of SEQ ID NO:4 or its RNA equivalent;
e) have a sequence which is complementary to the sequences of d);
f) have a sequence which codes for a KIAA0659 polypeptide;
g) have a sequence which shows substantial identity with any of those of d), e) and f); or
h) is a fragment of d), e), f) or g), which is at least 10 nucleotides in length; and may have one or more of the following characteristics:
1) they may be DNA or RNA;
2) they may be single or double stranded;
3) they may be in substantially pure form. Thus, they may be provided in a form which is substantially free from contaminating proteins and/or from other nucleic acids; and
4) they may be with introns or without introns (e.g. as cDNA).

Fragments of KIAA0659 nucleic acids are preferably at least 20, at least 30, at least 50, at least 100 or at least 250 nucleotides in length.

The invention also provides the use of nucleic acids which are complementary to the KIAA0659 nucleic acids described in (d)-(h) above, and can hybridise to said KIAA0659 nucleic acids. Such nucleic acid molecules are referred to as “hybridising” nucleic acid molecules. For example, but without limitation, hybridising nucleic acid molecules can be useful as probes or primers. Hybridising nucleic acid molecules may have a high degree of sequence identity along its length with a nucleic acid molecule within the scope of (d)-(h) above (e.g. at least 50%, at least 75%, at least 80%, at least 85%, at least 90%, at least 95%, or at least 98% sequence identity). The use of hybridising nucleic acid molecules that can hybridise to any of the nucleic acid molecules discussed above, e.g. in hybridising assays, is also covered by the present invention.

Hybridisation assays can be used for screening, prognosis, diagnosis, or monitoring of therapy of carcinoma in a subject. Accordingly, such a hybridisation assay comprises:
i) contacting a biological sample, obtained from a subject, containing nucleic acid with a nucleic acid probe capable of hybridising to a KIAA0659 nucleic acid molecule, under conditions such that hybridisation can occur; and
ii) detecting or measuring any resulting hybridisation.

Preferably, such hybridising molecules are at least 10 nucleotides in length and are preferably at least 25 or at least 50 nucleotides in length. More preferably, the hybridising nucleic acid molecules specifically hybridise to nucleic acids within the scope of any one of (d) to (h), above. Most preferably, the hybridisation occurs under stringent hybridisation conditions. One example of stringent hybridisation conditions is where attempted hybridisation is carried out at a temperature of from about 35° C. to about 65° C. using a salt solution which is about 0.9M. However, the skilled person will be able to vary such conditions as appropriate in order to take into account variables such as probe length, base composition, type of ions present, etc.

The invention also provides a diagnostic kit comprising a nucleic acid probe capable of hybridising to RNA encoding a KIAA0659 polypeptide, suitable reagents and instructions for use.

In a further embodiment, a diagnostic kit is provided comprising in one or more containers a pair of primers that under appropriate reaction conditions can prime amplification of at least a portion of a KIAA0659 nucleic acid molecule, such as by polymerase chain reaction (see e.g. Innis et al., 1990, PCR Protocols, Academic Press, Inc., San Diego, Calif.), ligase chain reaction (see EP 320,308) use of Qβ replicase, cyclic probe reaction, or other methods known in the art. Typically, primers are at least eight nucleotides long and will preferably be at least ten to twenty-five nucleotides long and more preferably fifteen to twenty-five nucleotides long. In some cases, primers of at least thirty or at least thirty-five nucleotides in length may be used.

In yet another aspect, the present invention provides the use of at least one KIAA0659 nucleic acid in the manufacture of a medicament for use in the treatment and/or prophylaxis of carcinoma.

In a specific embodiment, hybridising KIAA0659 nucleic acid molecules are used as anti-sense molecules, to alter the expression of KIAA0659 polypeptides by binding to complementary KIAA0659 nucleic acids and can be used in the treatment and/or prophylaxis or prevention of carcinoma. An antisense nucleic acid includes a KIAA0659 nucleic acid capable of hybridising by virtue of some sequence complementarity to a portion of an RNA (preferably mRNA) encoding a KIAA0659 polypeptide. The antisense nucleic acid can be complementary to a coding and/or non-coding region of an mRNA encoding such a polypeptide. Most preferably, expression of a KIAA0659 polypeptide is inhibited by use of antisense nucleic acids. Thus, the present invention provides the therapeutic or prophylactic use of nucleic acids comprising at least eight nucleotides that are antisense to a gene or cDNA encoding a KIAA0659 polypeptide.

In another embodiment, symptoms of carcinoma may be ameliorated by decreasing the level or activity of a KIAA0659 polypeptide by using gene sequences encoding a polypeptide as defined herein in conjunction with well-known gene “knock-out,” ribozyme or triple helix methods to decrease gene expression of the polypeptide. In this approach, ribozyme or triple helix molecules are used to modulate the activity, expression or synthesis of the gene, and thus to ameliorate the symptoms of carcinoma. Such molecules may be designed to reduce or inhibit expression of a mutant or non-mutant target gene. Techniques for the production and use of such molecules are well known to those of skill in the art.

Endogenous KIAA0659 polypeptide expression can also be reduced by inactivating or “knocking out” the gene encoding the polypeptide, or the promoter of such a gene, using s targeted homologous recombination (e.g. see Smithies, et al., 1985, Nature 317:230-234; Thomas & Capecchi, 1987, Cell 51:503-512; Thompson et al., 1989, Cell 5:313-321; and Zijlstra et al., 1989, Nature 342:435-438). For example, a mutant gene encoding a non-functional polypeptide (or a completely unrelated DNA sequence) flanked by DNA homologous to the endogenous KIAA0659 gene (either the coding regions or regulatory regions of the gene encoding the polypeptide) can be used, with or without a selectable marker and/or a negative selectable marker, to transfect cells that express the target gene in vivo. Insertion of the DNA construct, via targeted homologous recombination, results in inactivation of the target gene.

In another embodiment, the nucleic acid is administered via gene therapy (see for example Hoshida, T. et al., 2002, Pancreas, 25:111-121; Ikuno, Y. 2002, Invest. Ophthalmol. Vis. Sci. 2002 43:2406-2411; Bollard, C., 2002, Blood 99:3179-3187; Lee E., 2001, Mol. Med. 7:773-782). Gene therapy refers to administration to a subject of an expressed or expressible KIAA0659 nucleic acid. Any of the methods for gene therapy available in the art can be used according to the present invention.

Delivery of the therapeutic KIAA0659 nucleic acid into a patient can be direct in vivo gene therapy (i.e. the patient is directly exposed to the nucleic acid or nucleic acid-containing vector) or indirect ex vivo gene therapy (i.e. cells are first transformed with the nucleic acid in vitro and then transplanted into the patient).

For example for in vivo gene therapy, an expression vector containing the KIAA0659 nucleic acid is administered in such a manner that it becomes intracellular, i.e. by infection using a defective or attenuated retroviral or other viral vectors as described, for example, in U.S. Pat. No. 4,980,286 or by Robbins et al., 1998, Pharmacol. Ther. 80:35-47.

The various retroviral vectors that are known in the art are such as those described in Miller et al. (1993, Meth. Enzymol. 217:581-599) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek (1999, Human Gene Therapy, 10:2451-2459). Chimeric viral vectors that can be used are those described by Reynolds et al. (1999, Molecular Medicine Today, 1:25-31). Hybrid vectors can also be used and are described by Jacoby et al. (1997, Gene Therapy, 4:1282-1283).

Direct injection of naked DNA or through the use of microparticle bombardment (e.g. Gene Gun®; Biolistic, Dupont) or by coating it with lipids can also be used in gene therapy. Cell-surface receptors/transfecting compounds or through encapsulation in liposomes, microparticles or microcapsules or by administering the nucleic acid in linkage to a peptide which is known to enter the nucleus or by administering it in linkage to a ligand predisposed to receptor-mediated endocytosis (See Wu & Wu, 1987, J. Biol. Chem., 262:4429-4432) can be used to target cell types which specifically express the receptors of interest.

In another embodiment a nucleic acid ligand compound comprising a KIAA0659 nucleic acid can be produced in which the ligand comprises a fusogenic viral peptide designed so as to disrupt endosomes, thus allowing the KIAA0659 nucleic acid to avoid subsequent lysosomal degradation. The KIAA0659 nucleic acid can be targeted in vivo for cell specific endocytosis and expression by targeting a specific receptor such as that described in WO92/06180, WO93/14188 and WO 93/20221. Alternatively the nucleic acid can be introduced intracellularly and incorporated within the host cell genome for expression by homologous recombination (See Zijlstra et al, 1989, Nature, 342:435-428).

In ex vivo gene therapy, a gene is transferred into cells in vitro using tissue culture and the cells are delivered to the patient by various methods such as injecting subcutaneously, application of the cells into a skin graft and the intravenous injection of recombinant blood cells such as haematopoietic stem or progenitor cells.

Cells into which a KIAA0659 nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryotcytes, granulocytes, haematopoietic cells or progenitor cells, and the like.

In a one aspect, the pharmaceutical composition comprises a KIAA0659 nucleic acid, said nucleic acid being part of an expression vector that expresses a KIAA0659 polypeptide or chimeric protein thereof in a suitable host. In particular, such a nucleic acid has a promoter operably linked to the polypeptide coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller & Smithies, 1989, Proc. Natl. Acad. Sci. USA 86:8932-8935; Zijlstra et al., 1989, Nature 342:435-438).

KIAA0659 nucleic acids may be obtained using standard cloning and screening techniques, from a cDNA library derived from mRNA in human cells, using expressed sequence tag (EST) analysis (Adams, M. et al., 1991, Science, 252:1651-1656; Adams, M. et al., 1992, Nature 355:632-634; Adams, M. et al., 1995, Nature, 377:Suppl: 3-174). KIAA0659 nucleic acids can also be obtained from natural sources such as genomic DNA libraries or can be synthesized using well known and commercially available techniques. The KIAA0659 nucleic acids comprising coding sequence for KIAA0659 polypeptides described above can be used for the recombinant production of said polypeptides. The KIAA0659 nucleic acids may include the coding sequence for the mature polypeptide, by itself; or the coding sequence for the mature polypeptide in reading frame with other coding sequences, such as those encoding a leader or secretory sequence, a pre-, pro- or prepro-protein sequence, a cleavable sequence or other fusion peptide portions, such as an affinity tag or an additional sequence conferring stability during production of the polypeptide. Preferred affinity tags include multiple histidine residues (for example see Gentz et al., 1989, Proc. Natl. Acad. Sci USA 86:821-824), a FLAG tag, HA tag or myc tag. The KIAA0659 nucleic acids may also contain non-coding 5′ and 3′ sequences, such as transcribed, non-translated sequences, splicing and polyadenylation signals, ribosome binding sites and sequences that stabilize mRNA.

KIAA0659 polypeptide derivatives, above, can be created by introducing one or more nucleotide substitutions, additions or deletions into the nucleotide sequence of a KIAA0659 nucleic acid such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues.

A KIAA0659 nucleic acid encoding a KIAA0659 polypeptide, including homologues and orthologues from species other than human, may be obtained by a process which comprises the steps of screening an appropriate library under stringent hybridisation conditions with a labelled probe having the sequence of a KIAA0659 nucleic acid as described in (d)-(h) above, and isolating full-length cDNA and genomic clones containing said nucleic acid sequence. Such hybridisation techniques are well-known in the art. One example of stringent hybridisation conditions is where attempted hybridisation is carried out at a temperature of from about 35° C. to about 65° C. using a salt solution of about 0.9M. However, the skilled person will be able to vary such conditions as appropriate in order to take into account variables such as probe length, base composition, type of ions present, etc. For a high degree of selectivity, relatively stringent conditions such as low salt or high temperature conditions, are used to form the duplexes. Highly stringent conditions include hybridisation to filter-bound DNA in 0.5M NaHPO₄, 7% sodium dodecyl sulphate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & Sons, Inc., New York, at p. 2.10.3). For some applications, less stringent conditions for duplex formation are required. Moderately stringent conditions include washing in 0.2×SSC/0.1% SDS at 42° C. (Ausubel et al., 1989, supra). Hybridisation conditions can also be rendered more stringent by the addition of increasing amounts of formamide, to destabilise the hybrid duplex. Thus, particular hybridisation conditions can be readily manipulated, and will generally be chosen as appropriate. In general, convenient hybridisation temperatures in the presence of 50% formamide are: 42° C. for a probe which is 95-100% identical to the fragment of a gene encoding a polypeptide as defined herein, 37° C. for 90-95% identity and 32° C. for 70-90% identity.

One skilled in the art will understand that, in many cases, an isolated cDNA sequence will be incomplete, in that the region coding for the polypeptide is cut short at the 5′ end of the cDNA. This is a consequence of reverse transcriptase, an enzyme with inherently low processivity (a measure of the ability of the enzyme to remain attached to the template during the polymerization reaction), failing to complete a DNA copy of the mRNA template during 1^{st} strand cDNA synthesis.

Methods to obtain full length cDNAs or to extend short cDNAs are well known in the art, for example RACE (Rapid amplification of cDNA ends; e.g. Frohman et al., 1988, Proc. Natl. Acad. Sci USA 85:8998-9002). Recent modifications of the technique, exemplified by the Marathon™ technology (Clontech Laboratories Inc.) have significantly simplified the search for longer cDNAs. This technology uses cDNAs prepared from mRNA extracted from a chosen tissue followed by the ligation of an adaptor sequence onto each end. PCR is then carried out to amplify the missing 5′-end of the cDNA using a combination of gene specific and adaptor specific oligonucleotide primers. The PCR reaction is then repeated using nested primers which have been designed to anneal with the amplified product, typically an adaptor specific primer that anneals further 3′ in the adaptor sequence and a gene specific primer that anneals further 5′ in the known gene sequence. The products of this reaction can then be analysed by DNA sequencing and a full length cDNA constructed either by joining the product directly to the existing cDNA to give a complete sequence, or carrying out a separate full length PCR using the new sequence information for the design of the 5′ primer.

A further aspect of the invention relates to a vaccine composition of use in the treatment and/or prophylaxis of carcinoma. A KIAA0659 polypeptide or nucleic acid as described above can be used in the production of vaccines for treatment and/or prophylaxis of carcinoma. Such material can be antigenic and/or immunogenic. Antigenic includes a protein or nucleic acid that is capable of being used to raise antibodies or indeed is capable of inducing an antibody response in a subject. Immunogenic material includes a protein or nucleic acid that is capable of eliciting an immune response in a subject. Thus, in the latter case, the protein or nucleic acid may be capable of not only generating an antibody response but, in addition, a non-antibody based immune responses, i.e. a cellular or humoral response. It is well known in the art that is possible to identify those regions of an antigenic or immunogenic polypeptide that are responsible for the antigenicity or immunogenicity of said polypeptide, i.e. an epitope or epitopes. Amino acid and peptide characteristics well known to the skilled person can be used to predict the antigenic index (a measure of the probability that a region is antigenic) of a KIAA0659 polypeptide. For example, but without limitation, the ‘Peptidestructure’ program (Jameson and Wolf, 1988, CABIOS, 4(1):181) and a technique referred to as ‘Threading’ (Altuvia Y. et al., 1995, J. Mol. Biol. 249:244) can be used. Thus, the KIAA0659 polypeptides may include one or more such epitopes or be sufficiently similar to such regions so as to retain their antigenic/immunogenic properties.

Since a polypeptide or a nucleic acid may be broken down in the stomach, the vaccine composition is preferably administered parenterally (e.g. subcutaneous, intramuscular, intravenous or intradermal injection).

Accordingly, in further embodiments, the present invention provides:
a) the use of such a vaccine in inducing an immune response in a subject; and
b) a method for the treatment and/or prophylaxis of carcinoma in a subject, or of vaccinating a subject against carcinoma which comprises the step of administering to the subject an effective amount of a KIAA0659 polypeptide or nucleic acid, preferably as a vaccine.

Preferred features of each embodiment of the invention are as for each of the other embodiments mutatis mutandis. All publications, including but not limited to patents and patent applications cited in this specification are herein incorporated by reference as if each individual publication were specifically and individually indicated to be incorporated by reference herein as though fully set forth.

The invention will now be described with reference to the following examples, which are merely illustrative and should not in any way be construed as limiting the scope of the present invention.

FIG. 1 shows the amino acid sequences of a KIAA0659 polypeptide (SEQ ID NO:1). Tandem mass spectrum peptides are shown in bold and underlined, mass matches are in bold italics.

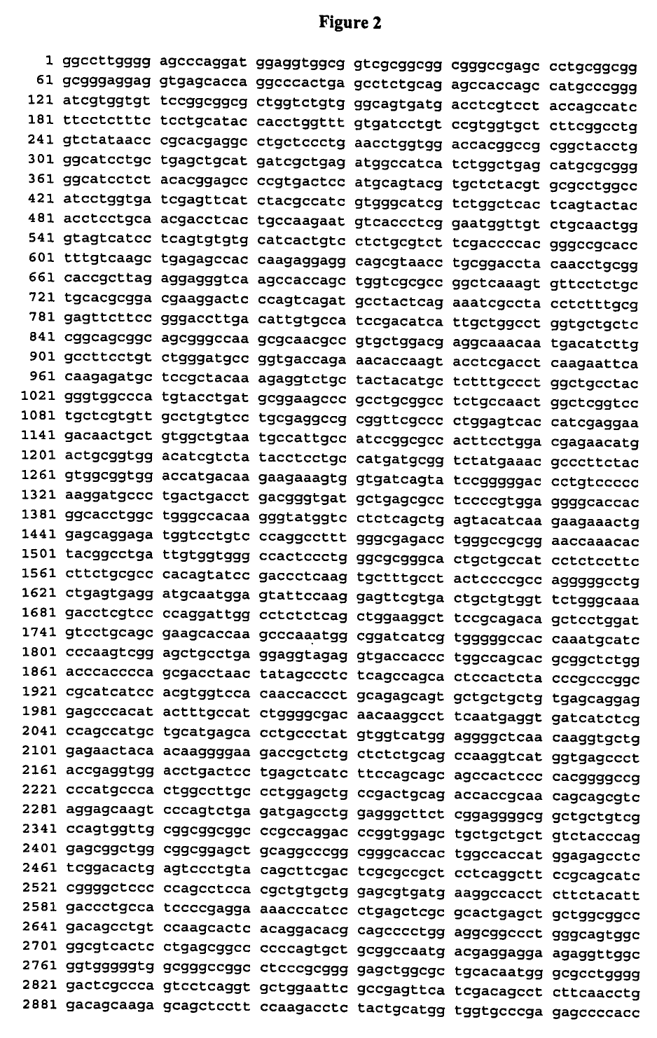

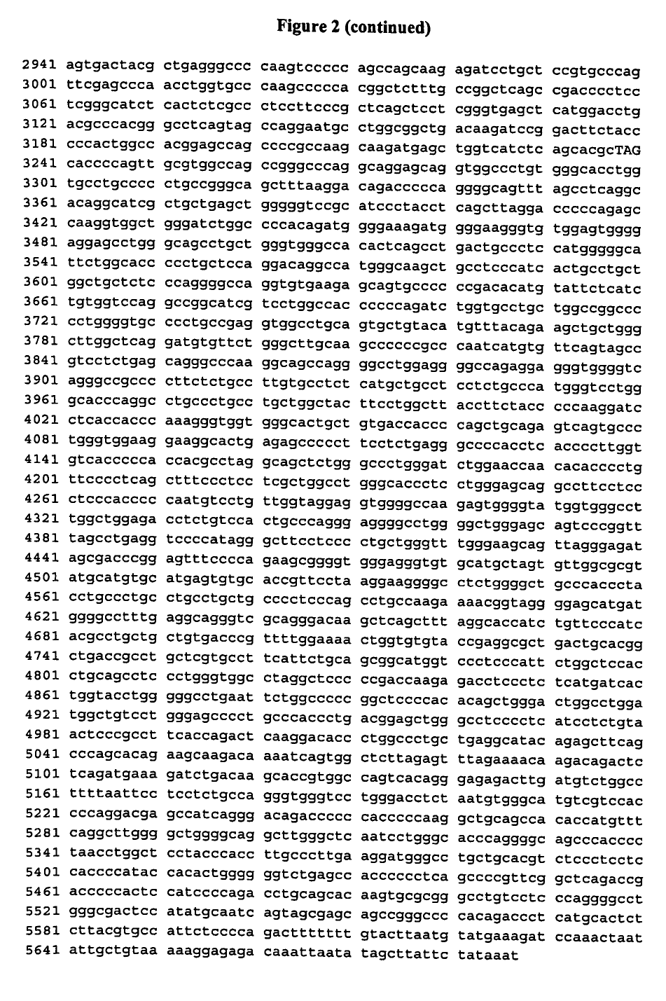

FIG. 2 shows the corresponding nucleic acid sequence (SEQ ID NO:2) of the KIAA0659 polypeptide of FIG. 1.

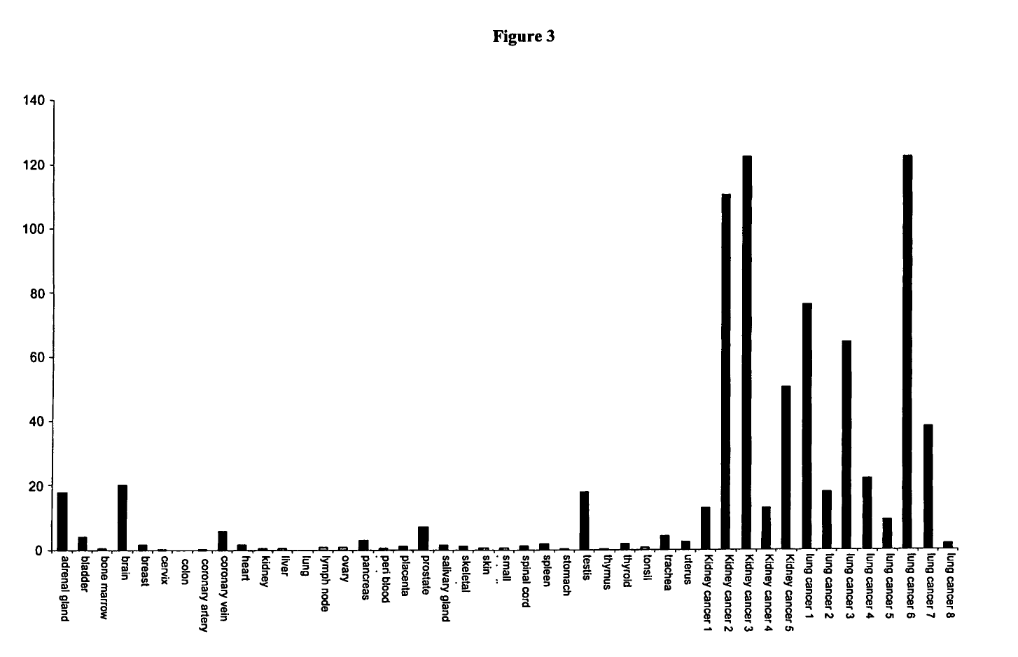

FIG. 3 shows the distribution of KIAA0659 mRNA in normal tissues, kidney cancer and lung cancer samples; mRNA levels were quantified by real time RT-PCR and are expressed as the number of copies ng⁻¹ cDNA.

### EXAMPLE 1

### Isolation of KIAA0659 Protein from HepG2 Cancer Cell Lines

Proteins in HepG2 cancer cell line membranes were separated by SDS-PAGE and analysed.

1a—Cell Culture

The human hepatocellular carcinoma cells, HepG2 cells (ECACC Cat No. 85011430) were cultured in EMEM+1% NEAA+10% FBS. Cells were grown at 37° C. in a humidified atmosphere of 95% air and 5% carbon dioxide.

1b—Cell Fractionation and Plasma Membrane Generation

Purified membrane preparations were isolated from the cell lines. Adherent cells (2×10⁸) were washed three times with PBS and scraped using a plastic cell lifter. Cells were centrifuged at 1000×g for 5 min at 4° C. and the cell pellet was resuspended in homogenisation buffer (250 mM Sucrose, 10 mM HEPES, 1 mM EDTA, 1 mM Vanadate and 0.02% azide, protease inhibitors). Cells were fractionated using a ball bearing homogeniser (8.002 mm ball, HGM Lab equipment) until approx. 95% of cells were broken. Membranes were fractionated using the method described by Pasquali et al (Pasquali C. et al., 1999 J. Chromatography 722: pp 89-102). The fractionated cells were centrifuged at 3000×g for 10 min at 4° C. and the postnuclear supernatant was layered onto a 60% sucrose cushion and centrifuged at 100 000×g for 45 min. The membranes were collected using a pasteur pipette and layered on a preformed 15 to 60% sucrose gradient and spun at 100 000×g for 17 hrs. Proteins from the fractionated sucrose gradient were run on a 4-20% 1D gel (Novex) and subjected to Western blotting.

1c—Preparation of Plasma Membrane Fractions for 1D-gel Analysis

Plasma membrane fractions that had transferrin immunoreactivity but no oxidoreductase II or calnexin immunoreactivity were identified and pooled. This pool which represented the plasma membrane fraction was diluted at least four times with 10 mM HEPES, 1 mM EDTA 1 mM Vanadate, 0.02% Azide and added to a SW40 or SW60 tube and centrifuged at 100 000×g for 45 min with slow acceleration and deceleration. The supernatant was removed from the resulting membrane pellet and the pellet washed three times with PBS-CM. The membrane pellet was solubilised in 2% SDS in 63 mM TrisHCl, pH 7.4. A protein assay was performed followed by the addition of mercaptoethanol (2% final), glycerol (10%) and bromophenol blue (0.0025% final) was added. A final protein concentration of 1 microgram/microlitre was used for 1D-gel loading.

1d—1D-Gel Technology

Protein or membrane pellets were solubilised in 1D-sample buffer (approximately 1 mg/ml) and the mixture heated to 95° C. for 5 min.

Samples were separated using 1D-gel electrophoresis on pre-cast 8-16% gradient gels purchased from Bio-Rad (Bio-Rad Laboratories, Hemel Hempstead, UK). A sample containing 30-50 micrograms of the protein mixtures obtained from a detergent extract were applied to the stacking gel wells using a micro-pipette. A well containing molecular weight markers (10, 15, 25, 37, 50, 75, 100, 150 and 250 kDa) was included for calibration by interpolation of the separating gel after imaging. Separation of the proteins was performed by applying a current of 30 mA to the gel for approximately 5 hrs or until the bromophenol blue marker dye had reached the bottom of the gel.

After electrophoresis the gel plates were prised open, the gel placed in a tray of fixer (10% acetic acid, 40% ethanol, 50% water) and shaken overnight. The gel was then primed for 30 minutes by shaking in a primer solution (7.5% acetic acid, 0.05% SDS in Milli-Q water) followed by incubation with a fluorescent dye (0.06% dye in 7.5% acetic acid) with shaking for 3 hrs. A preferred fluorescent dye is disclosed in U.S. Pat. No. 6,335,446. Sypro Red (Molecular Probes, Inc., Eugene, Oreg.) is a suitable alternative dye for this purpose.

A digital image of the stained gel was obtained by scanning on a Storm Scanner (Molecular Dynamics Inc, USA) in the blue fluorescence mode. The captured image was used to determine the area of the gel to excise for in-gel proteolysis.

1e—Recovery and Analysis of Selected Proteins

Each vertical lane of the gel was excised using a stainless steel scalpel blade, then sliced horizontally every 0.5 mm along the length of the protein-stained area. Each 0.5 mm slice was subjected to in-gel digestion with trypsin (Modified trypsin, Promega, Wis., USA) to generate tryptic digest peptides. Recovered samples were divided into two. Prior to MALDI analysis samples were desalted and concentrated using C18 Zip Tips™ (Millipore, Bedford, Mass.). Samples for tandem mass spectrometry were purified using a nano LC system (LC Packings, Amsterdam, The Netherlands) incorporating C18 SPE material. Recovered s peptide pools were analysed by MALDI-TOF-mass spectrometry (Voyager STR, Applied Biosystems, Framingham, Mass.) using a 337 nm wavelength laser for desorption and the reflectron mode of analysis. Pools were also analyzed by nano-LC tandem mass spectrometry (LC/MS/MS) using a Micromass Quadrupole Time-of-Flight (Q-TOF) mass spectrometer (Micromass, Altrincham, UK). For partial amino acid sequencing and identification of membrane proteins uninterpreted tandem mass spectra of tryptic peptides were searched against a database of public domain proteins constructed of protein entries in the non-redundant database held by the National Centre for Biotechnology Information (NCBI) which is accessible at http://www.ncbi.nlm.nih.gov/using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662). The method described in WO 02/21139 was also used to interpret mass spectra.

Tandem mass spectra were found to match the GenBank accession number BAA31634.2 (see FIG. 1). These data suggest that the polypeptide described as KIAA0659 is likely to be of utility as a liver cancer target.

### EXAMPLE 2

### Normal Tissue Distribution and Disease Tissue Upregulation of KIAA0659 Using Quantitative RT-PCR (Taqman) Analysis

Real time RT-PCR was used to quantitatively measure KIAA0659 expression in a range of tumour tissues and matched controls. The kidney and lung tumour samples were obtained from Ardais Corp. (Lexington, Mass.) and Clinomics Biosciences Inc., MD. The primers used for PCR were as follows:
Sense, 5′-cgacctcactgccaagaatgtc-3′, (SEQ ID NO:3)
Antisense, 5′-cttgaccctcctctaagcggtg-3′ (SEQ ID NO:4)

Reactions containing 5 ng cDNA, SYBR green sequence detection reagents (PE Biosystems) and sense and antisense primers were assayed on an ABI7700 sequence detection system (PE Biosystems). The PCR conditions were 1 cycle at 50° C. for 2 min, 1 cycle at 95° C. for 10 min, and 40 cycles of 95° C. for 1 min, 65° C. for 30 sec. The accumulation of PCR product was measured in real time as the increase in SYBR green fluorescence, and the data were analysed using the Sequence Detector program v1.6.3 (PE Biosystems). Standard curves relating initial template copy number to fluorescence and amplification cycle were generated using the amplified PCR product as a template, and were used to calculate KIAA0659 copy number in each sample.

Relatively low expression levels of KIAA0659 were seen in normal tissues (FIG. 3) with the highest expression in normal adrenal, brain and testis. However, elevated expression was seen in all kidney and lung clinical tumour samples indicating that KIAA0659 is of utility as target for therapeutic intervention in kidney and lung cancers.

### EXAMPLE 3

### Cellular Localisation of KIAA0659 in Lung Carcinoma Cells

Immunocytochemical analysis of A549 cells transiently transfected with a mammalian expression vector encoding a KIAA0659 polypeptide was used to determine the membrane topology of KIAA0659. The expression plasmid was constructed by amplifying the KIAA0659 ORF from a plasmid template (KIAA0659 in pCR4-topo) using Pfu DNA polymerase (PfuTurbo Hotstart DNA polymerase, Stratagene) and the following primers: KIAA0659 forward 5′-ccataagcttgccaccatgcccgggatcgtggtgttcc-3′ (SEQ ID NO:5) and KIAA0659 reverse 5′-ccatggtaccgagcgtgctgagatgaccagctca-3′ (SEQ ID NO:6). The PCR product was digested with Hind III and Kpn I restriction endonucleases and cloned into p3×FLAG-CMV-14 vector (Sigma) digested with the same restriction endonucleases, such that the 3×FLAG epitope tag encoded by the vector was in frame with the 3′ end of the KIAA0659 ORF. KIAA0659 protein expressed from this plasmid was thus tagged at the C-terminus with 3 FLAG epitopes.

A549 cells were seeded into 8-well chamber slides, maintained at 37° C. in a humidified atmosphere of 95% air and 5% CO₂ for 24 hr and then transfected with the KIAA0659 expression plasmid using GeneJuice transfection reagent (Novagen) according to the manufacturer's instructions. Transfected cells were cultured overnight, washed with PBS, fixed with 4% paraformaldehyde and blocked with 5% donkey serum/PBS prior to immunocytochemical analysis with a biotin-conjugated anti-FLAG antibody (Sigma). Cells were either permeabilised with 0.1% saponin after fixation and before the addition of primary antibodies, to allow the detection of intracellular FLAG-tagged protein, or left unpermeabilised to detect FLAG epitopes exposed on the cell surface. The cells were then incubated with the biotinylated anti-FLAG antibody or rabbit IgG as control. Following a 1 hr incubation at room temperature with the primary antibodies, the cells were washed with 5% donkeyserum/PBS, incubated with ExtrAvidin-Cy3 (Sigma) for 30 min at room temperature, and then processed for fluorescence microscopy.

Anti-FLAG-specific plasma membrane staining was seen on A549 cells that were transfected with the expression plasmid encoding KIAA0659. No staining was observed on untransfected cells, or transfected cells incubated with the rabbit IgG antibody. Both permeabilised and non-permeabilised transfected cells stained with the anti-FLAG antibody. These data indicate that KIAA0659 is a plasma membrane protein with an extracellular C-terminal domain.

These data suggest that KIAA0659 is expressed in liver cancer-derived cell lines and shows increased expression in kidney and lung cancers. Additionally, KIAA0659 is shown to be exposed on the surface of cells. These data indicate that KIAA0659 is of utility as a marker for diagnosis of, and a target for therapeutic intervention in kidney, liver and lung cancers.

1. (canceled)

2. (canceled)

3. (canceled)

4. (canceled)

5. (canceled)

6. (canceled)

7. A method for the treatment and/or prophylaxis of carcinoma comprising administering a therapeutically effective amount of an agent which interacts with or modulates the expression or activity of a KIAA0659 polypeptide.

8. The method according to claim 7, wherein the agent is an antibody, functionally-active fragment, derivative or analogue thereof.

9. The method according to claim 8, wherein the antibody is monoclonal, polyclonal, chimeric, humanised or bispecific, or is conjugated to a therapeutic moiety, detectable label, second antibody or a fragment thereof, an effector or reporter molecule, a cytotoxic agent or cytokine.

10. A method for the treatment and/or prophylaxis of carcinoma comprising administering a therapeutically effective amount of a composition comprising a KIAA0659 polypeptide.

11. The method according to claim 10, wherein the composition is a vaccine.

12. The method according to claim 7, wherein the KIAA0659 polypeptide:
(a) comprises or consists of the amino acid sequence of SEQ ID NO:1; or
(b) is a derivative having one or more amino acid substitutions, modifications, deletions or insertions relative to the amino acid sequence of SEQ ID NO:1 which retains the activity of the KIAA0659 polypeptide.

13. A method of screening for anti-carcinoma agents that interact with a KIAA0659 polypeptide, said method comprising:
(a) contacting said polypeptide with a candidate agent; and
(b) determining whether or not the candidate agent interacts with said polypeptide.

14. The method according to claim 13, wherein the determination of an interaction between the candidate agent and KIAA0659 polypeptide comprises quantitatively detecting binding of the candidate agent and said polypeptide.

15. A method of screening for anti-carcinoma agents that modulate the expression or activity of a KIAA0659 polypeptide comprising:
(i) comparing the expression or activity of said polypeptide in the presence of a candidate agent with the expression or activity of said polypeptide in the absence of the candidate agent or in the presence of a control agent; and
(ii) determining whether the candidate agent causes the expression or activity of said polypeptide to change.

16. The method according to claim 15, wherein the expression or activity of said polypeptide is compared with a predetermined reference range.

17. The method according to claim 15, wherein part (ii) additionally comprises selecting an agent which interacts with or modulates the expression or activity of said polypeptide for further testing, or therapeutic or prophylactic use as an anti-carcinoma agent.

18. An agent identified by the method of claim 13, which interacts with or causes the expression or activity of said polypeptide to change.

19. A method of screening for and/or diagnosis or prognosis of carcinoma in a subject, and/or monitoring the effectiveness of carcinoma therapy, which comprises the step of detecting and/or quantifying in a biological sample obtained from said subject, the expression of a KIAA0659 polypeptide.

20. The method according to claim 19, wherein the expression of said polypeptide is compared to a previously determined reference range or control.

21. The method according to claim 19, wherein the step of detecting comprises:
(a) contacting the sample with a capture reagent that is specific for a KIAA0659 polypeptide; and
(b) detecting whether binding has occurred between the capture reagent and said polypeptide in the sample.

22. The method according to claim 21, wherein step (b) comprises detecting the captured polypeptide using a directly or indirectly labelled detection reagent.

23. The method according to claim 21, wherein the capture reagent is immobilised on a solid phase.

24. The method according to claim 13, wherein the polypeptide is detected and/or quantified using an antibody that specifically binds to a KIAA0659 polypeptide.

25. The method according to claim 24, wherein the antibody is conjugated to a detectable label, or a second antibody or a fragment thereof.

26. A diagnostic kit comprising a capture reagent specific for a KIAA0659 polypeptide, reagents and instructions for use.

27. (canceled)
